(19) United States
(12) Patent Application Publication
(10) Pub. No.: US 2013/0123854 A1
(43) Pub. Date: May 16, 2013

(54) SYSTEM AND METHOD FOR SPINAL STABILIZATION THROUGH MUTLI-HEAD SPINAL SCREWS

(71) Applicants: Dimitriy G. Kondrashov, San Francisco, CA (US); Jeremi M. Leasure, Oakland, CA (US); Grigoriy Kondrashev, Rockaway Beach, NY (US)

(72) Inventors: Dimitriy G. Kondrashov, San Francisco, CA (US); Jeremi M. Leasure, Oakland, CA (US); Grigoriy Kondrashev, Rockaway Beach, NY (US)

(21) Appl. No.: 13/678,353

(22) Filed: Nov. 15, 2012

Related U.S. Application Data
(60) Provisional application No. 61/560,391, filed on Nov. 16, 2011.

(51) Int. Cl. A61B 17/70

(52) U.S. Cl. CPC A61B 17/7032
USPC 606/264

(57) ABSTRACT

Embodiments of present invention disclose spinal screws having first and second heads for a versatile screw-rod construct to achieve spinal stabilization for patients with unstable spines. The first and second heads are used to connect first and second rods at the same bone anchor location. The first and second heads can accept rods of different diameter, materials and/or stiffness in order to accommodate the anatomical needs of the spine of a patient. Further, the first and second heads can positioned side by side to accommodate parallel rods or perpendicular to each other to accommodate rods with cross link rods. Further, the position of the first and second heads can be adjusted relative to each other.

### CLAIM OF PRIORITY

This present application claims the benefit of priority to U.S. Provisional Patent Application No. 61/560,391, filed Nov. 16, 2011 entitled “Multi-head Polyaxial Screw for Spine Surgery” which is incorporated herein by reference in its entirety.

### TECHNICAL FIELD

Embodiments of the present invention relate to systems and methods to stabilize a spinal column. More particularly, embodiments of the present invention relate to systems and methods for interconnecting spinal bone screws and rods in a spinal stabilization system.

### BACKGROUND OF INVENTION

A healthy spine plays vital roles in daily activity. It provides the torso support and the motion flexibility, such as flexion and extension in the anatomical frontal plane.

However, spinal diseases are very common and may inflict tremendous pain on the patients. Statistic shows that more than 65 million Americans suffer from lower back pain annually. And by the age of fifty, more than 80 percent of the population show different degrees of Degenerative Disc Disease (DDD). Other spinal diseases that cause pain and affect movement are, for example, spinal stenosis, tumors, scoliosis, deformities, and fractures. In many of these diseases, the unstable spine, due to a degenerated disc or a spinal defect, generates pressure on the nerves and causes pain. One effective remedy for these diseases is implanting a spinal stabilization system to support the patient's spine.

Usually, a spinal stabilization system consists of a combination of anchors, e.g., bolts, hooks, and/or screws; longitudinal members, e.g., plates, rods; and/or transverse connectors. And the spinal stabilization system can be either static (rigid) or dynamic.

A static (rigid) spinal stabilization system can be applied in a surgery called spinal fusion, in which vertebra bones are joined or healed together. Spinal screws and supporting rods are used to hold the vertebra bones stable until fusion occurs. However, due to the rigidness of fused vertebra bones, the adjacent intervertebrae discs often suffer from further degeneration and cause more complications.

In recent years, dynamic spinal stabilization systems are preferred over the static stabilization system. Instead of immobilizing the spine and destroying the anatomical structure, the dynamic spinal stabilization system aims to preserve much of the spinal anatomy and spinal motion. In a dynamic spinal stabilization system, polyaxial spinal screws are often employed to assist in bone screw and rod placement. Also, the supporting rods may employ elastic materials, such as polyethylene terephthalate (PET), to preserve the patient's anatomical structure motion.

Nonetheless, both static and dynamic spinal stabilization systems can utilize multiple supporting rods to provide spinal stabilization. And spinal screws are used to secure the multiple supporting rods to the vertebrae. Furthermore, spinal screws include pedicle screws in the thoracic or lumbar spine and lateral mass screws in the cervical spine.

Pedicle and lateral mass screws have become the main forms of fixation in the modern spine surgery. They offer multiple advantages, including three-column fixation, unparalleled ability to manipulate the spine in cases of spinal deformities, significant pullout strength and increased fusion rates, to name a few.

As indicated above, pedicle screws can have a rigid head—so-called monoaxial screws, as well as a rotating and angulating head—so-called polyaxial screws. Both screw designs can accept only one rod by the nature of its design.

In some cases, one may wish to place more than one rod into the same screw. First, sometimes it is desirable to run rods of different diameter or material at different levels to provide a more gradual transition from the stiffer fusion to the flexible unfused spine. This can potentially minimize the stresses on the adjacent vertebral segment and decrease the incidence of adjacent level degeneration. Second, one may want to transition from a bigger (e.g. 6.35 mm rod) system in the lumbar spine to the smaller (e.g. 5.5 mm rod) system in the thoracic spine for deformity correction surgery. This can potentially limit the stiffness at the top of the construct and decrease the incidence of proximal junctional kyphosis. Third, in some revision cases, when the exposure of the entire old construct is unnecessary, one may want to connect the top or bottom of the existing construct without removing the entire old rod. Fourth, sometimes one may desire to run more than two rods in cases of highly unstable spine. For instance, vertebral column resection and pedicle subtraction osteotomy at times require running three or four rods in parallel, because of a high chance of failure of two rods only.

The current options for running more than one rod through the same screw are quite limited. One can run three or four rods in parallel by utilizing either cross-links or domino-type connector. Alternatively, one can utilize wires or hooks as the alternate anchor at the same vertebral level and run a hook-based rod. Those options tend to be cumbersome with limited strength and rigidity of the resultant construct.

### SUMMARY OF THE INVENTION

Embodiments of the present invention comprise multi-head spinal screw systems that address the aforementioned problems.

Embodiments of the present invention that are configured to be embedded in a patient vertebra comprise a bone screw shank that has a proximal end and a distal end, with the distal end adapted to be embedded in the vertebra of a patient. The embodiment further comprises a first head mounted on the proximal end of the screw shank with the first head including a first channel that can receive a spinal rod. The system further comprises a second head connected to the first head, the second head including a second channel to receive a second rod.

In one embodiment of the invention the first head is a polyaxial head and the second head can be selectable positionable or moveable relative to the first head.

In another embodiment of the invention, a third head can be secured to the first head, wherein the third head includes a third channel for receiving a third rod.

In yet another embodiment of the invention, the first channel is adapted to receive a first rod of a first diameter and the second channel is adapted to receive a second rod of a second diameter. Preferably, the first and second rods have different stiffness and flexibility characteristics such that a transition from a more rigid section of the spine, due to for example a fusion, can be made to a more normal, flexible section of the spine.

Still further in another embodiment of the invention, the first channel can be adapted to receive a first rod of a first material and the second channel can be adapted to receive a second rod of a second material, wherein the first material and the second material can have different stiffness and flexibility characteristics. With such an embodiment, again a gradual transition can be made from a stiffer portion of the spine to a more flexible section of the spine.

In another embodiment of the invention the system can include the first head, the second head secured to the first head and first, and second rods of the same or different diameters and materials.

In still another embodiment of the invention, the first head can be adapted to receive a rod running in a first direction, and the second head can be adapted to receive a rod running in a second direction. In another embodiment of the invention, the first and second rods can run in the same direction or can run in for example in directions such that the rods are perpendicular to each other or at an angle other than parallel to each other. For crosslinking a first spinal system to a second spinal system the second head can accept a second rod which is for example oriented at an angle to the first rod accepted by the first head.

In another embodiment of the invention, a method comprises implanting a screw shank into the bone of a patient, inserting a first rod in a first head associated with the screw shank and inserting a second rod associated with the first head. Embodiments of the method include adjusting the position of the first head relative to the second head and also using a first rod that has different stiffness or flexibility characteristics from the second head in order for example to make a transition from a stiffer portion of the spine of the patient to a more flexible portion of the spine.

Accordingly, embodiments of the present invention can be used when multiple supporting rods need to be linked together to provide the requisite force in a spinal stabilization system. For example, in a spinal fusion surgery, it may be beneficial to use supporting rods of different diameters or materials at the fused vertebrae and the unfused vertebrae. Such a construct may provide gradual transition from the stiffer fused vertebrae to the flexible unfused vertebrae. As a result, it alleviates the stresses on the adjacent intervertebrae discs and reduces adjacent discs degenerations.

By way of a more specific example, in a spine deformity correction surgery, embodiments of the invention may be used to connect a bigger (e.g. 6.35 mm) supporting rod in the lumbar spine and transition to a small (e.g. 5.5 mm) supporting rod in the thoracic spine. Such a construct may potentially reduce the rigidity at the thoracic spine and decrease the incidence of proximal junctional kyphosis.

Yet in another example, embodiments of the present invention can be used in a revision surgery, when the exposure of the entire original construct is not necessary. In such situations, it is desirable to connect the top or bottom of the original rods without removing the entire original construct.

Still in another example, in case of a substantially unstable spine, such as vertebral column resection and pedicle subtraction osteotomy, embodiments of the invention may be used to run more than two or more supporting rods in parallel to provide sufficient stabilization force.

Further advantages and objects of embodiments of the invention can be obtained from a review of the specification, claims and figures.

### BRIEF DESCRIPTION OF THE DRAWINGS

FIG. 1 is a perspective view of a spinal screw-rod system in accordance with an embodiment of the present invention.

FIG. 2 is a perspective view of a spinal screw-rod system in accordance with an embodiment of the present invention that provides two spinal screw-rods constructs to stable the spine.

FIG. 3 is a perspective view of a spinal screw-rod system in accordance with an embodiment of the present invention that provides three parallel supporting rods at the same anchor point.

FIG. 4 is a perspective view of a spinal screw-rod system in accordance with another embodiment of the present invention for connecting different rods at the same anchor point.

FIGS. 5A, 5B and 5C include a perspective, a partial top view and a top posterior view representing a spinal screw-rod system in accordance with an embodiment of the present invention that provides cross-linked supporting rods.

FIG. 6 depicts a method of an embodiment of the invention.

### DETAILED DESCRIPTION OF THE PREFERRED EMBODIMENTS

Embodiments of the present invention relate to a system and method for rigid or dynamic spinal stabilization through multi-head spinal screws. Embodiments of the present invention may be used in other orthopedic surgery to provide an incorporated bone anchor and rod connector.

In the following description, the invention will be illustrated by way of example and not by way of limitation in the figures of the accompanying drawings. References to various embodiments in this disclosure are not necessarily to the same embodiment, and such references mean at least one. While specific implementations are discussed, it is understood that this is provided for illustrative purposes only. A person skilled in the relevant art will recognize that other components and configurations may be used without departing from the scope and spirit of the invention.

Furthermore, in certain instances, numerous specific details will be set forth to provide a thorough description of the invention. However, it will be apparent to those skilled in the art that the invention may be practiced without these specific details. In other instances, well-known features have not been described in as much detail as not to obscure the invention.

Embodiments of the present invention disclose spinal screws as the anchoring member for the screw-rod construct. However, a practitioner with ordinary skill in the art may use other anchoring devices with multiple heads, such as plates, hooks, claws or wires, to replace spinal screws.

Furthermore, embodiments of the present invention may employ multiple types of spinal screws, including pedicle screws in the thoracic or lumbar spine and lateral mass screws in the cervical spine.

According to one embodiment of the present invention, a spinal screw may employ a polyaxial mechanism to allow certain degree of placement mobility between the screw shank and the screw head. A monoaxial spinal screw has one axis, which means that the screw head forms a linear and rigid structure with the screw shank. The linear and rigid structure may generate a stressful connections between the spinal screw and the supporting rod and often results in complications, such as degeneration of the adjacent intervertebrae discs. In contrast, a polyaxial spinal screw has multiple axes, which means that the screw shank can swivel freely against the screw head prior to, for example, the polyaxial head being locked to the rod and the screw shank. This structure may reduce vertebral stress because the position of the rods and the two or more spinal screws can be adjusted before the spinal system is locked into position.

According to one embodiment of the present invention, spinal screws may employ various biocompatible materials to provide the necessary strength and versatility. In one embodiment, the spinal screw may consist of titanium for its resistance to corrosion, endurability, and compatibility with MRI (Magnetic Resonance Imaging) machines. In another embodiment, the spinal screw may employ alloys such as 316L stainless steel, 316LVM stainless steel, 22Cr-13Ni-5Mn stainless steel, Ti-6Al-4V, which allows the surgeon to build an implant system to fit the anatomical and physiological requirements of the patient. Yet in another embodiment, the spinal screw may employ different materials for the screw shank and screw heads.

According to one embodiment of the present invention, a spinal screw may connect supporting rods of different sizes at one anchor point. For example, in a spine deformity correction surgery, sometimes it is necessary to employ a bigger (e.g. 6.35 mm) supporting rod in the lumbar spine and transition to a small (e.g. 5.5 mm) supporting rod in the thoracic spine. Such a construct may potentially reduce the rigidity at the thoracic spine and decrease the incidence of proximal junctional kyphosis. In another example, in a spinal fusion surgery, sometimes the screw-rod construct may need to provide more gradual transition from the stiffer fused vertebrae to the flexible unfused vertebrae through employing different diameters of supporting rods. Such a construct alleviates the stresses on the adjacent intervertebrae discs and reduces adjacent discs degenerations.

To connect supporting rods of different sizes, according to one embodiment of the present invention, a spinal screw may employ a first screw head having a first channel and a second screw head having a second channel, wherein the first channel has different diameter from the second channel. For example, the first head includes a channel for a 6.35 mm rod in the lumbar spine, and the second head includes a channel for a 5.5 mm rod in the thoracic spine. In another embodiment, a spinal screw may adopt different colors to code the different screw heads for easier recognition. Yet in another embodiment, the sizes of the two or more screw heads may be different to accommodate to different supporting rods.

According to one embodiment of the present invention, a spinal screw may connect supporting rods of different biocompatible materials at one anchor point. For example, in a spinal fusion surgery, sometimes the screw-rod construct may need to provide more gradual transition from the stiffer fused vertebrae to the flexible unfused vertebrae through employing different materials for the supporting rods, i.e. a more elastic rod material at the flexible unfused vertebrae. Such a construct alleviates the stresses on the adjacent intervertebrae discs and reduces adjacent discs degenerations. In another example, the supporting rod may employ elastic and resilient material to allow it slightly extending and adapting to spine motions, in accordance with the philosophy of dynamic spinal stabilization.

Furthermore, biocompatible materials may be employed by the supporting rod include, but not limited to, polyethylene terephthalate (PET), Nitinol (NiTi), Ti alloys, as well as other elastic metal alloys or polymers, including polyetheretherketone (PEEK), polyetherketoneketone (PEKK), polyetherketone (PEK), polyetherketoneetherketone-ketone (PEKEKK), and polyetheretherketoneketone (PEEKK).

According to one embodiment of the present invention, a multi-head spinal screw may be used to connect supporting rods which are parallel or perpendicular to each other. Particularly, the two-head spinal screw may function as cross-connector for the cross-linked rods, which would facilitate the process of cross-linking, eliminate several moving parts, decrease the potential for loosing connection or instrument slippage, as well as possibly reduce costs.

For example, in case of a substantially unstable spine, such as vertebral column resection and pedicle subtraction osteotomy, sometimes it is necessary to run more than two or more supporting rods in parallel to provide sufficient stabilization force. In another example, perpendicular rods, or cross-linked rods, are necessary to add torsional rigidity to the rod-screw construct.

To connect two supporting rods which are parallel to each other, according to one embodiment of the present invention, a spinal screw may employ a first screw head having a first channel and a second screw head having a second channel, wherein the first channel is parallel to the second channel. Similarly, to connect two supporting rods which are perpendicular to each other, according to one embodiment of the present invention, the first channel of the first screw head is perpendicular to the second channel of the second screw head. Furthermore, to connect three parallel supporting rods, a spinal screw may employ a third screw head having a third channel which is parallel to the other two parallel channels. In such embodiments, the first head can be rigidly secured to a second head by, for example, a laser welding technique, a welding technique or a adhesive or bonding technique. In other embodiments the first head can be machined with the second head out of a piece of material. Still further the first head may be cased, molded or formed together with a second head.

According to one embodiment of the present invention, a spinal screw with multiple heads may employ a mechanism to allow relative movement and positioning between the two or more heads before, for example, the heads are locked together. In such situations a first head can be movably or positionable relative to a second head. Such a mechanism can comprise locking splines where a spline associated with a first head can be received and locked to a spline in a second head. The position of the first head relative to the second head can be adjusted, by for example rotating the first head relative to the second head before the first spline is mated and locked to the second spline. Previously, because of the fixed and rigid connection between supporting rods and/or spinal screws, a surgeon often needs to bend the supporting rods to contour to the anatomical and physiological requirements of the patient. Such bending of the rods can be reduced as the polyaxial heads can move relative to each other, and relative to the bone screw shanks and the rods to accommodate the anatomy and bone structure of the patient.

In one embodiment, such a rod bending procedure may be replaced by adjusting the relative angles of the supporting rods at the connected heads. In one embodiment, the surgeon first implants the multi-head spinal screw in the patient's vertebra bone, and places the two or more supporting rods into each head of a multi-head spinal screw. And before or after placement of the rods, the surgeon adjusts the relative angles of the heads to allow the supporting rods to accommodate the anatomical and physiological requirements of the patient.

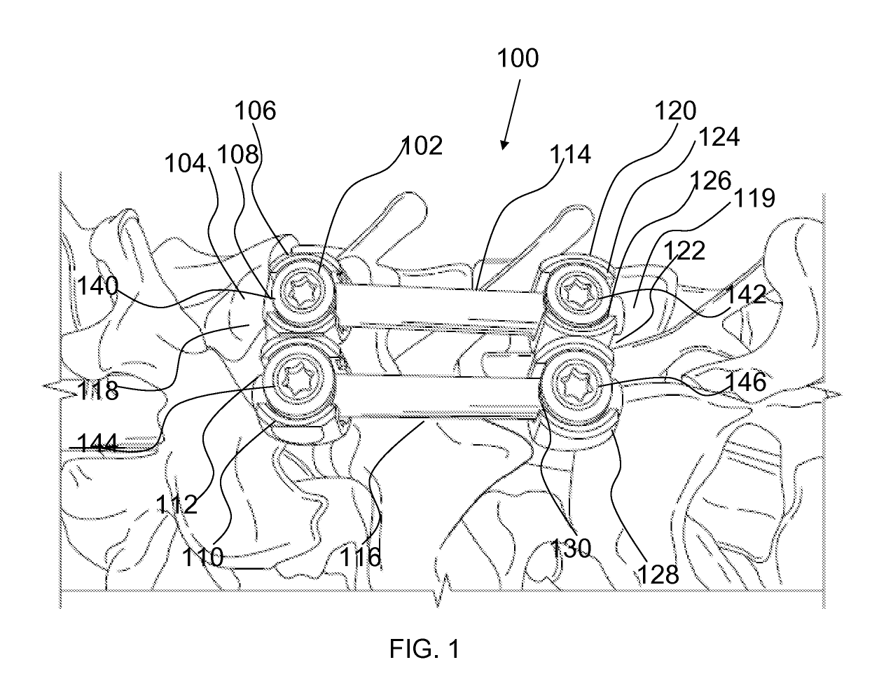

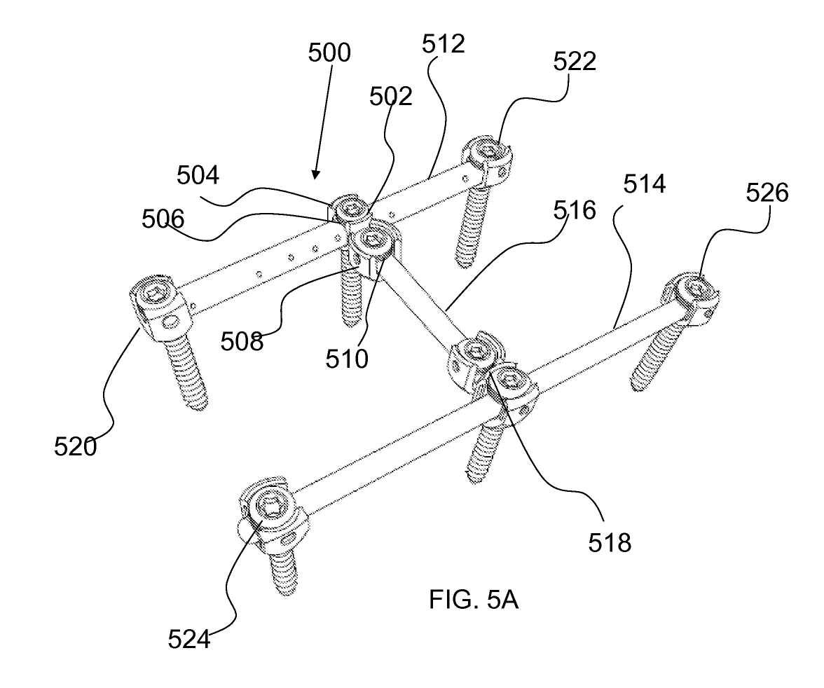

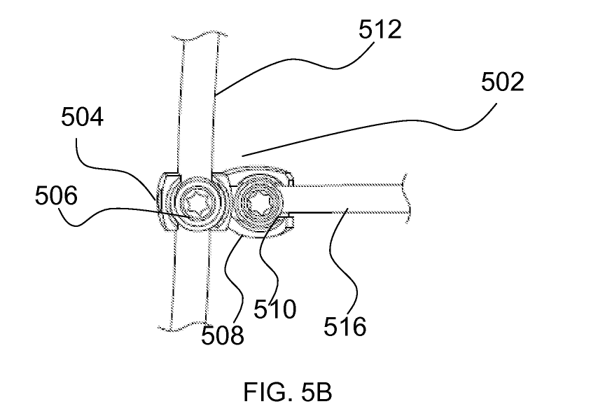

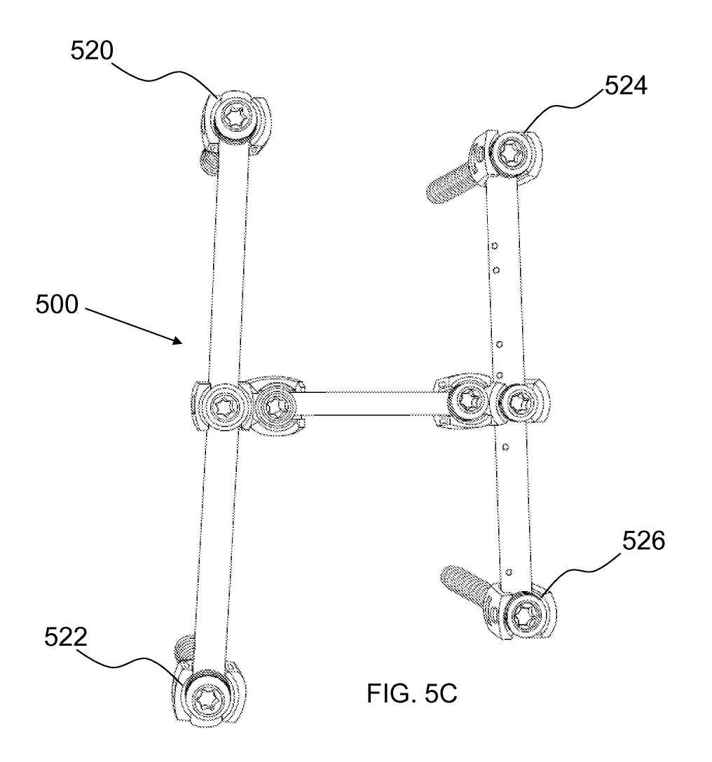

Referring now to FIG. 1, FIG. 1 is a perspective view of a spinal screw-rod construct in accordance with an embodiment of the present invention. In one embodiment, a spinal screw-rod construct 100 may comprise two two-head spinal screws 102, 120, each including a screw shank 104, 122 (embedded in bone in FIG. 1 and similar to the screw shank as seen in FIG. 5A), a first head 106, 124 having a first channel 108, 126, a second head 110, 128 having a second channel 112, 130, a first rod 114, and a second rod 116. In one embodiment, the two-head spinal screws 102 and 120 may employ a polyaxial mechanism.

In one embodiment, when the patient's spine needs to be stabilized, such as in a spinal fusion surgery, the screw shanks 104 and 122 are first implanted in a patient's adjacent vertebra 118, 119 at a predetermined distance. Then the first rod 114 is inserted into the first channels 108 and 126, and the second rod 116 is inserted into the second channels 112 and 128. Set screws 140,142 are then used to lock the rod 114 in the respective polyaxial heads 106,124 and to lock the respective polyaxial heads to the bone screw shanks 104,122. Set screws 144, 146 are used to lock the other rod 116 into the respective heads 110, 128. In an embodiment where heads 106 and 110 and heads 126, 128 can move relative to each other, prior to implantation of the screw shanks into the bone, the heads are appropriately positioned relative to each other. In an alternative method the heads are moved relative to each other prior to and during the placement of the rods in the heads. Here, the first rod 114 is about parallel to the second rod 116 to provide reinforced stabilization to the spine. In another embodiment, a rod locking element may be employed in the screw head to lock the supporting rod.

Accordingly, in the embodiment of FIG. 1, the embodiment includes a multi-headed pedicle or lateral mass screws. The number of heads per screw can be either two or three. The polyaxial first heads 106, 124 are used to position the heads in relation to the screw shanks. The second head is connected to the first head and rotates either with it (rigid connection) or has some degree of free motion compared to the first head (flexible connection).

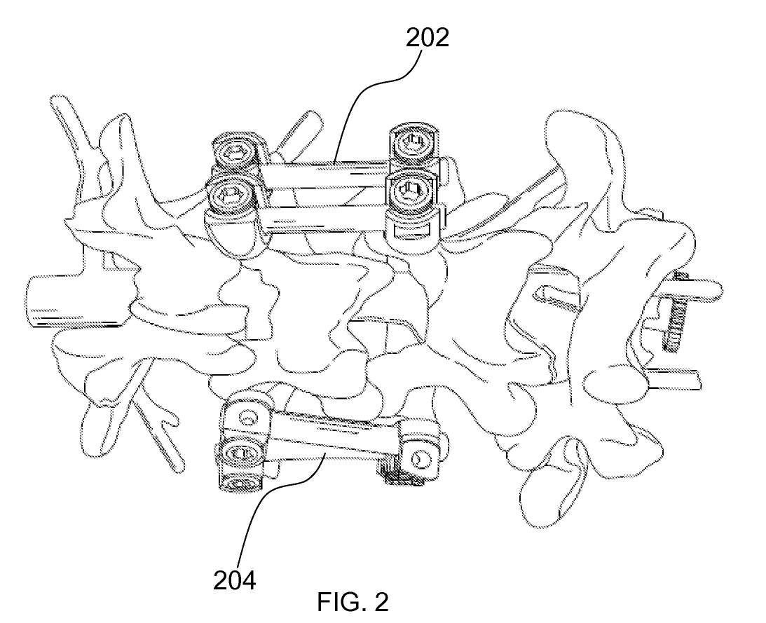

FIG. 2 is a perspective view of a spinal screw-rod system in accordance with an embodiment of the present invention that employs two spinal screw-rod constructs to provide spinal stabilization. In this embodiment, two spinal screw-rod constructs 202, 204 (similar to the construct in FIG. 1) may be implanted in parallel at the posterior segments of the patient's vertebral column.

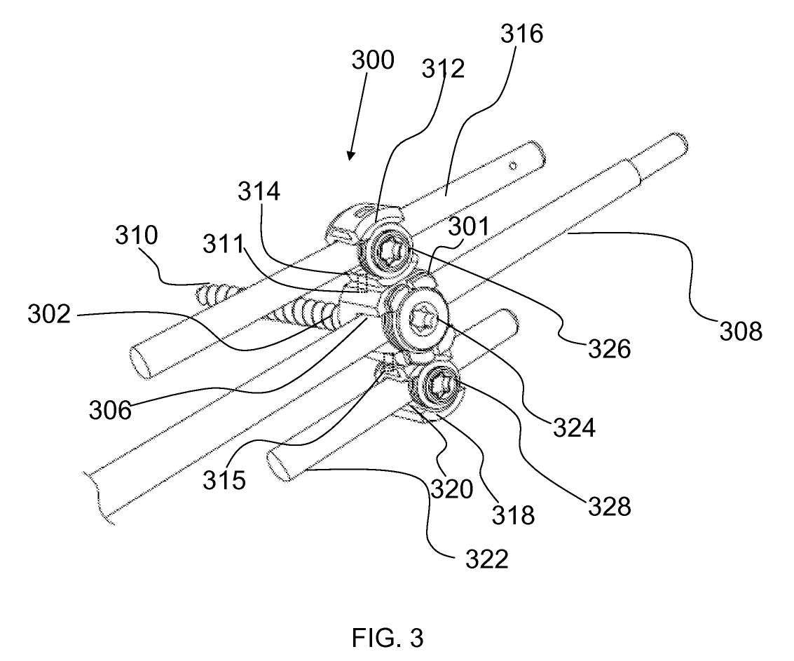

FIG. 3 is a perspective view of a spinal screw-rod system in accordance with an embodiment of the present invention that provides three parallel supporting rods at the same anchor point. In one embodiment, a spinal screw-rod construct 300 with three parallel rods may provide more support to an extremely unstable spine. As FIG. 3 illustrates, a three-head spinal screw 302 may align a first rod 308, a second rod 316, and a third rod 322 at one anchor point.

In one embodiment, the three-head spinal screw 302 with screw shank 310 may comprise a first head 301, a second head 312, and a third head 318, which are rigidly connected to each other.

In another embodiment, one or more of the additional heads 312, 318 may be movably connected to the first head 301, which allows the surgeon to adjust the supporting rods and heads, as previously explained, to contour to the anatomical and physiological requirements of the patient. Set screws 324,326, and 328 can be used to lock the rods in the respective heads 301, 312 and 318.

As for the mechanism to realize the relative movement between the two or more heads, one may utilize a mechanical mechanism which permits relative movement between two connected mechanical parts, in this case, two screw heads. For example, the two or more screw heads may be connected through a spline mechanism with mating teeth 311, 315, which allows them to move relative to each other. Alternatively, the spline mechanism 311, 315 can be replaced with a pin. Thus, the heads are pinned together with one or both of the heads rotatable about the pin. Thus, heads 312 and 318 may be movable relative to head 301.

In one embodiment, the three-head spinal screw 302 may employ a polyaxial mechanism between the first head 301 and the screw shank 310.

In another embodiment, the three-head spinal screw 302 may connect supporting rods of different sizes. As FIG. 3 illustrates, in one embodiment, the first head 301 may include a first channel 306, the second head 312 may include a second channel 314, and the third head 318 may include a third channel 320. And the sizes of the three channels 306, 314 and 320 may be various to receive supporting rods with different diameters. Yet in another embodiment, the three-head spinal screw 302 may connect supporting rods made of different materials.

As with FIGS. 1 and 2, the embodiment of FIG. 3 allows multiple rods to be connected, which rods may be the same or different diameters and/or be made of the same or different materials to be secured at the same anchor point which in this case is where the bone screw shank is secured into the bone of the patient.

An embodiment with three side by side rods may be used, for example, in situations such as a spondylectomy for tumors, and unstable fractures or dislocations.

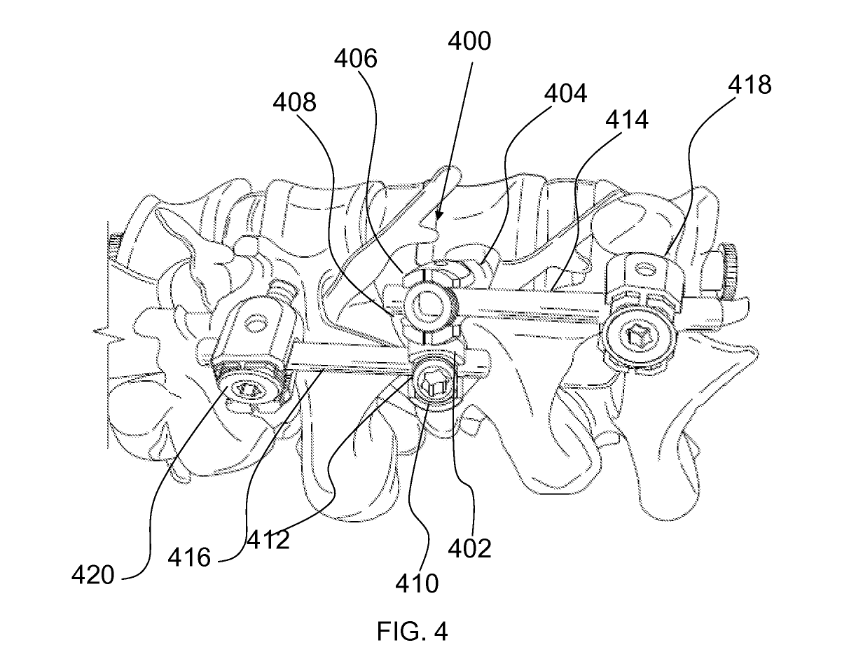

FIG. 4 is a perspective view of a spinal screw-rod system in accordance with another embodiment of the present invention for constructing, rods end-to-end, at the same anchor point. In one embodiment, a spinal screw-rod construct 400 with end-to-end rods may be used to provide a more gradual transition of flexibility or stiffness along the vertebral column. Such gradual transition of flexibility or stiffness may be realized through a rod-to-rod connection of different supporting rods, either in sizes or in material characteristics.

For example, in a spine deformity correction surgery, it may be desirable to employ a bigger (e.g. 6.35 mm) supporting rod in the lumbar spine and transition to a small (e.g. 5.5 mm) supporting rod in the thoracic spine. Such a construct may potentially reduce the rigidity at the thoracic spine and decrease the incidence of proximal junctional kyphosis. In another example, in a spinal fusion surgery, the screw-rod construct may desirably provide gradual transition from the stiffer fused vertebrae to the flexible unfused vertebrae through employing different diameters of supporting rods. Similar transitional force can also be achieved through employing supporting rods of different materials, i.e. a more elastic rod material at the flexible unfused vertebrae. Such a construct alleviates the stresses on the adjacent intervertebrae discs and reduces adjacent discs degenerations.

As FIG. 4 depicts, according to one embodiment of the spinal screw-rod construct 400, a two-head spinal screw 402 is placed between two mono-head spinal polyaxial screws 418, 420. The two-head spinal screw 402 includes two connected screw heads, a first polyaxial head 406 connected to a screw shank and a second head 410 connected to the first head 406. A first rod 414 is affixed by the first head 406 and the mono-head screw 418. And a second rod 416 is affixed by the second head 410 and the mono-head screw 420. As aforementioned, the first rod 414 and the second rod 416 may be different in sizes and material characteristics to deliver a more dynamic and transient force along the vertebral column.

In one embodiment, the first head 406 may be rigidly connected to the second head 410. In another embodiment, the first head 406 may be movably connected to the second head 410.

FIGS. 5A and 5C are different perspective views of a spinal screw-rod system in accordance with an embodiment of the present invention that provides cross-linked supporting rods. FIG. 5B is a partial top view of FIG. 5A. FIG. 5C is a top posterior view of the embodiment 500.

Referring now to FIG. 5A, a spinal screw-rod construct 500 with cross-linked rods may be used when additional torsional rigidity is required in the spinal stabilization system. The two-head spinal screw may function as cross-connector for the cross-linked rods, which facilitates the process of cross-linking, eliminates several moving parts, decreases the potential for loosing connection or instrument slippage, as well as possibly reduces costs.

In FIG. 5A, in one embodiment, two conventional supporting rods 512, 514 are affixed to the vertebrae through preferably four polyaxial mono-head spinal screws 520, 522, 524, 526 and two two-head spinal screws 502, 518. A transverse supporting rod 516 is cross-linked between the two conventional supporting rods 512, 514 through the two-head spinal screws 502, 518.

FIG. 5B is an top partial view of the cross-linked connection between the conventional supporting rod 512 and the transverse supporting rod 516. In one embodiment, the first channel 506 of the first head 504 is perpendicular to the second channel 510 of the second head 508. Thus, the conventional supporting rod 512 which is affixed by the first channel 506 is also perpendicular to the transverse supporting rod 516 which is affixed by the second channel 510.

In one embodiment, the transverse supporting rod 516 and conventional supporting rods 512, 514 may be different in sizes and material characteristics to deliver a more dynamic and transient force along the vertebral column.

Yet in another embodiment, for the two-head spinal screw 502 (or 518), the first head 504 may be rigidly connected to the second head 508. Still in another embodiment, the first head 504 may be movably connected to the second head 508.

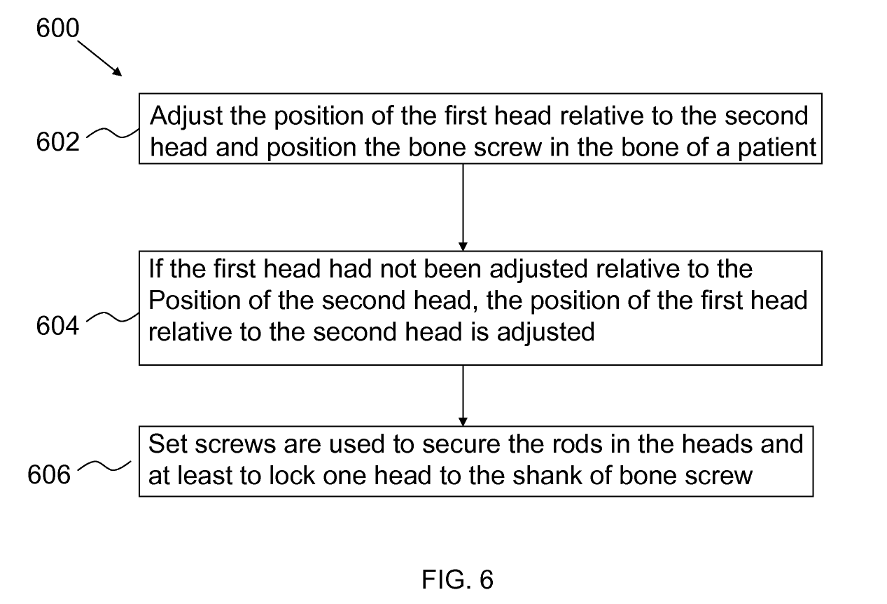

FIG. 6 illustrates an embodiment 600 of a method of the invention. In a method of an embodiment 600 of the invention, at 602 a screw shank with two or more heads is implanted in the bone of a patient and the first head is adjusted relative to the position of the second head. At 602, if the first head had not been adjusted relative to the position of the second head, the first head is adjusted relative to the second head after implantation of the bone screw shank in the bone of the patient. At step 604 first and second rods are positioned in the first and second heads. At step 606 set screws are used to secure the first and second rods in the first and second heads and at least one of the set screws is used to lock the first and second heads the shank of the bone screw.

The foregoing description of the preferred embodiments of the present invention has been provided for purposes of illustration and description. It is not intended to be exhaustive or to limit the invention to the precise forms disclosed. Many modifications and variations can be apparent to the practitioner skilled in the art. Embodiments were chosen and described in order to best explain the principles of the invention and its practical application, thereby enabling others skilled in the art to understand the invention. It is intended that the scope of the invention be defined by the following claims and their equivalents.

What is claimed is:

1. A spinal screw system configured to be embedded in a patient vertebra comprising:
a screw shank having a proximal end and a distal end, the distal end adapted to be embedded in the patient vertebra;
a first head mounted on the proximal end of the screw shank, the first head including a first channel adapted to receive a first rod, and
a second head connected to the first head, the second head including a second channel adapted to receive a second rod.

2. The spinal screw of claim 1 wherein the first head polyaxially mounts on the proximal end of the screw shank.

3. The spinal screw system of claim 1 wherein the first head is movably connected to the second head.

4. The spinal screw system of claim 1 wherein the first head is rigidly connected to the second head.

5. The spinal screw system of claim 1, further comprising a third head connected to the first head, the third head including a third channel adapted to receive a third rod.

6. The spinal screw system of claim 1 wherein the first channel is adapted to accept a rod of a first diameter and the second channel is adapted to accept a rod of a second diameter.

7. The spinal screw system of claim 1 wherein the first channel is adapted to accept a rod of a first material and the second channel is adapted to accept a rod of a second material.

8. The spinal screw system of claim 1 wherein the second channel is parallel to the first channel to receive parallel rods.

9. The spinal screw system of claim 1 wherein the second channel is perpendicular to the first channel to receive cross-linked rods.

10. The spinal screw system of claim 1 wherein the system comprises the first and second rods.

11. The spinal screw system of claim 1 including the first and second rods, wherein the first and second rods are about parallel to each other.

12. The spinal screw system of claim 1 including the first and second rods, wherein the first and second rods are about perpendicular to each other.

13. The spinal screw system of claim 5 including the first, second and third rods, wherein the first, second and third rods are about parallel to each other.

14. The spinal screw system of claim 1 wherein said first head is a polyaxial head and the second head is movably connected to the first head.

15. The spinal screw system of claim 1 including a second screw shank with a polyaxial head and a third screw shank with a polyaxial head and including the first rod connected between the first head and the head of the second screw shank and the second rod connected between the second head and the head of the third screw shank and wherein the first rod and the second rod have at least one different diameters, different materials, and/or different stiffness.

16. The spinal screw system of claim 15 wherein the first channel of the first head is one of about parallel and about perpendicular to the channel of the second head.

17. A method for implanting spinal screws configured to be embedded in a patient bone comprising:
receiving a screw shank having a proximal end and a distal end, the distal end configured to be embedded in the patient bone;
inserting a first rod into a first head mounted on the proximal end of the screw shank, the first head including a first channel configured to receive the first rod; and
inserting a second rod into a second head connected to the first head, the second head including a second channel to receive the second rod.

18. The method of claim 17 wherein the receiving step includes implanting the shank in the bone of the patient.

19. The method of claim 18 including the step of adjusting the position of the first head relative to the second head one or both of prior to implanting the bone shank in the bone or after the bone shank is implanted in the bone.

20. The method of claim 19 including the step of using set screws to lock the rods into the heads.

21. A spinal anchor system configured to be embedded in a patient vertebra comprising:
an anchor having a proximal end and a distal end, the distal end adapted to be embedded in the patient vertebra;
a first head mounted on the proximal end of the anchor, the first head including a first channel adapted to receive a first rod, and
a second head connected to the first head, the second head including a second channel adapted to receive a second rod.

22. The spinal anchor system of claim 21 wherein the first head polyaxially mounts on the proximal end of the screw shank.

23. The spinal anchor system of claim 21 wherein the first head is movably connected to the second head.

24. The spinal anchor system of claim 21 wherein the first head is rigidly connected to the second head.

25. The spinal anchor system of claim 21 including the first and second rods, wherein the first and second rods are about parallel to each other.

26. The spinal anchor system of claim 21 including the first and second rods, wherein the first and second rods are about perpendicular to each other.
